(19) United States
(12) Patent Application Publication
(10) Pub. No.: US 2009/0192608 A1
(43) Pub. Date: Jul. 30, 2009

(54) Method & apparatus for arthroscopic biceps tenodesis

(76) Inventor: Lonnie E. Paulos, Houston, TX (US)

Correspondence Address: JOHN BROOKS LAW LLC, P.O. Box 156, WRENTHAM, MA, 02093, US

(21) Appl. No.: 12/011,455

(22) Filed: Jan. 28, 2008

(51) Int. Cl. A61F 2/08; A61B 19/00

(52) U.S. Cl. 623/13.14; 128/898

(57) ABSTRACT

A method and apparatus for arthroscopic biceps tenodesis wherein the method involves arthroscopically determining that biceps tenodesis is desirable; arthroscopically separating the long head of the biceps tendon from bone attachment; creating and placing a wedge at the proximal end of the long head of the biceps tendon; and allowing the tendon to slide through the glenohumeral joint and the wedge structure to be wedged in the bicipital groove and wherein the apparatus includes a clamshell type, button wedge having a football shaped body with top and bottom portions hinged at one end and with locking structure at an opposite end to lock the top and bottom portions together, such that staggered ribs on the top portion and bottom portion grip top and bottom surfaces of the long head of the tendon when the top and bottom portions of the button wedge are clamped together.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO MICROFICHE APPENDIX

Not Applicable.

### BACKGROUND OF THE INVENTION

1. Field of the Invention

This invention relates to a method and apparatus for performing an arthroscopic biceps tenodesis.

2. Description of the Prior Art

With the increased use of systematic arthroscopic shoulder examinations and arthroscopic procedures, pathology of the long head of the biceps tendon has become more frequently identified. Biceps tendon pathology is found in a wide variety of shoulder conditions, ranging from instability to rotator cuff disorders and primary biceps tendon pathology. Prior to arthroscopic viewing of the glenohumeral joint and the detailed examination that it affords, the long head of the biceps was rarely visualized surgically and biceps tendon pathology was not recognized until in later stages. Arthroscopy allows earlier recognition and treatment of biceps pathology. While the definitive role of the long head of the biceps tendon remains controversial, its importance as a contributor of shoulder dysfunction has been increasingly recognized.

Treatment options for long head of the biceps pathology initially consists of observation or non-operative management during early stages or upon initial presentation. Known surgical options initially include debridement of frayed tendinous portions. Tenotomy treatment has usually been reserved for older, less active patients with chronic shoulder pain. In some cases, tenotomy has been used in connection with massive, irreparable rotator cuff tears. Tenodesis of the biceps tendon is commonly indicated for younger, more active patients because it affords a primary advantage of improved cosmesis.

Open techniques are well known and provide secure fixation for the transplanted biceps tendon. Such open techniques, however, have potential major disadvantages since morbidity may be incurred through necessary accessory incisions. More recently, some arthroscopic procedures have been proposed. The advantages of use of arthroscopic procedures over open techniques include decreased morbidity resulting from open incisions and more rapid rehabilitation. Disadvantages are the increased operative time required and, currently, a lack of knowledge of the long term success of the treatments.

It is well recognized that a known process identified as “keyhole” tenodesis, may be used and be performed either arthroscopically or open and is often used in the repair of rotator cuffs. In addition, it is sometimes used as an isolated procedure with what is called “interval lesions”. Furthermore, even isolated biceps pathology, on occasion, requires some form of tenodesis.

At the present time there is available, on the market, an interference screw that is sold for anchoring a tendon end to bone during keyhole biceps tenodesis. Unfortunately, the interference screw causes tendon damage and may amputate the tendon at the edge of the bone panel. The tendon then tends to degenerate. It is also becomes very difficult to even place sutures that will hold the tendon during healing. Consequently, it is necessary that during an operation, special suturing techniques be used to try and fix a degenerate tendon. The healing of a sutured tendon takes much longer and requires protection, with a resultant loss of motion and with a potential need for further surgery that will include follow-up scope evaluation and manipulation of the tendon.

### SUMMARY OF THE INVENTION

The present invention provides a new technique of arthroscopically-assisted biceps tenodesis, which, in general, becomes a “wedge” tenodesis. The procedure involves the isolation of a bicep tendon, tenotomy of the tendon at the long head of the biceps tendon, and creation of the placement of a wedging structure at the proximal end of the long head of the biceps tendon. The tendon is allowed to slide through the glenohumeral joint, where before entering the intertubecular groove of the humerus, the wedging structure is firmly lodged in the biciptal groove.

Objects of the Invention

Principal objects of the invention are to provide biceps tenodesis that is safe, effective and reproducible. Other objects are to provide a procedure that can be performed quickly and without the need for specialized equipment. At most, a very simple, easily used anchor button may be employed as the wedging structure placed at the proximal end of the long head of the biceps tendon. Accessory incisions are not required and operative time and costs are reduced in comparison with other possible procedures.

Features of the Invention

It is believed that the procedure of creating and using a wedging structure in biceps tenodesis can successfully improve pain control, while still resulting in minimal cosmetic deformity and/or loss of strength of the bicep.

Wedging tenodesis requires an initial arthroscopic assessment of the biceps tendon. If appropriate significant symptoms and pathology are found to be present, the wedging tenodesis may be performed. A grasping monofilament stitch is placed into the proximal aspect of the biceps tendon in known fashion, through a cannula that is typically placed in the rotator interval and immediately anterior to the AC joint. The suture is then retrieved through the cannula, i.e., an 8 mm cannula, placed through an accessory anterolateral portal. Thereafter, the long head of the biceps is released from its attachment on the superior labrum.

The released tendon is delivered into the anterolateral cannula and the anterolateral cannula is withdrawn, thus, pulling the tendon out the anterolateral portal. A clamp is placed across the tendon approximately three cm from its end to prevent it from prematurely retracting back into the shoulder.

A wedging structure is affixed to the remaining stump of tendon and in a preferred embodiment, the wedging structure may be fashioned from the stump end of the tendon by forming the end as a knot. The knot may be formed and tied as an overhead knot in the stump end of the tendon. Alternatively, a knot may be formed with braided suture securing a rolled top of the stump end. The knot formed in the tendon needs to be large enough to catch on the end of the intertubercular groove, in order to provide an effective tenodesis. The glenohumeral joint is then visualized arthroscopically from the posterior portal and the biceps tendon is allowed to retract into the intertubercular groove. The knot in the tendon secures it at the proximal aspect of the groove and prevents continued tendon retraction. After satisfactory placement has been confirmed, all suture strands are arthroscopically cut. The wedging tenodesis is performed in advance of other shoulder pathology, such as repair of rotator cuff tears.

Another embodiment of wedging tenodesis involves use of a clamp-type wedge in place of a formed knot, as previously described.

In this embodiment, the procedure is as previously described, except that rather than formation of a knot in the stub end of the tendon, a clamshell type clamp wedge is attached to the stub end of the tendon. While other types of anchors may be used, the clamshell type clamp wedge is the preferred anchor. The clamp wedge comprises a clamshell type button having an elongate, generally football shaped configuration.

The clamshell type button of the invention includes a top section and a bottom section connected at one end of the football shaped body by a hinge and with locking holding means at the opposite locking end of the body to secure the top and bottom sections together in a clamping configuration. A hole through which a traction suture is passed is formed in the hinge end of the bottom section. A rack having spaced teeth along its length is formed at the locking end of the bottom section. The rack pivots at the locking end of the bottom section to extend into and through an opening formed at the locking end of the top section. A shaped locking flange formed to extend into the opening at the locking end of the top section is sufficiently flexible to allow insertion of the rack into the opening and, because of the shape prevents withdrawal of the inserted rack. Once the rack is pushed into the opening the top and bottom sections are securely locked together. The long head end of the biceps tendon being repaired is positioned between the top and bottom sections and when the top and bottom portions are locked together, the clamshell type button is firmly secured to the tendon.

The outer surfaces of the top and bottom sections are preferably made “rough” to enhance wedging during use. In addition, staggered clamping ribs on the engaging surfaces of the top and bottom sections are pushed into the tendon to further insure secure attachment of the clamshell type button to the tendon and to prevent sliding of the button along and off the tendon.

Additional objects and features of the invention will become apparent to persons skilled in the art to which the invention pertains from the following detailed description and claims.

### BRIEF DESCRIPTION OF THE DRAWINGS

In the Drawings

FIG. 1 is a pictorial view of a long head of a biceps tendon arthoscopically end trimmed and pulled through a portal to be prepared for a wedge tenodesis;

FIG. 2, a pictorial view of a biceps tendon with a tied knot formed therein during wedge tenodesis;

FIG. 3, a similar view showing formation of a sutured knot formed in a biceps tendon during wedge tenodesis;

FIG. 4, a wedge tenodesis with a knot wedged in the bicipital groove;

FIG. 5, a pictorial view of a tenodesis having a knot formed in the end of a biceps tendon and showing the knot arranged to be wedged into a keyhole opening of the type commonly formed in the humeras in preparation for a “keyhole” tenodesis;

FIG. 6, a pictorial view showing how a knotted end of a biceps tendon is moved into a keyhole opening;

FIG. 7, a similar view showing the knotted end of a biceps tendon wedged into a keyhole opening;

FIG. 8, a view like that of FIG. 6, showing how the knotted end is positioned in a keyhole opening;

FIG. 9, a pictorial view showing securement of a clamshell type clamp wedge to a biceps tendon;

FIG. 10, a view like that of FIG. 9, showing a clamshell type button wedge clamped to a biceps tendon and wedged into a bicipital groove;

FIG. 11, a top plan view of the closed button wedge of FIG. 10;

FIG. 12, a side elevation view of the button wedge of FIG. 10, showing the wedge in a clamping, locked position;

FIG. 13, a longitudinal section taken on the line 13-13 of FIG. 11;

FIG. 14, a view like that of FIG. 12, but showing the button wedge in a partially open condition and with a biceps tendon laid over the bottom portion of the button wedge; and

FIG. 15, an enlarged sectional view taken on the line 15-15 of FIG. 9.

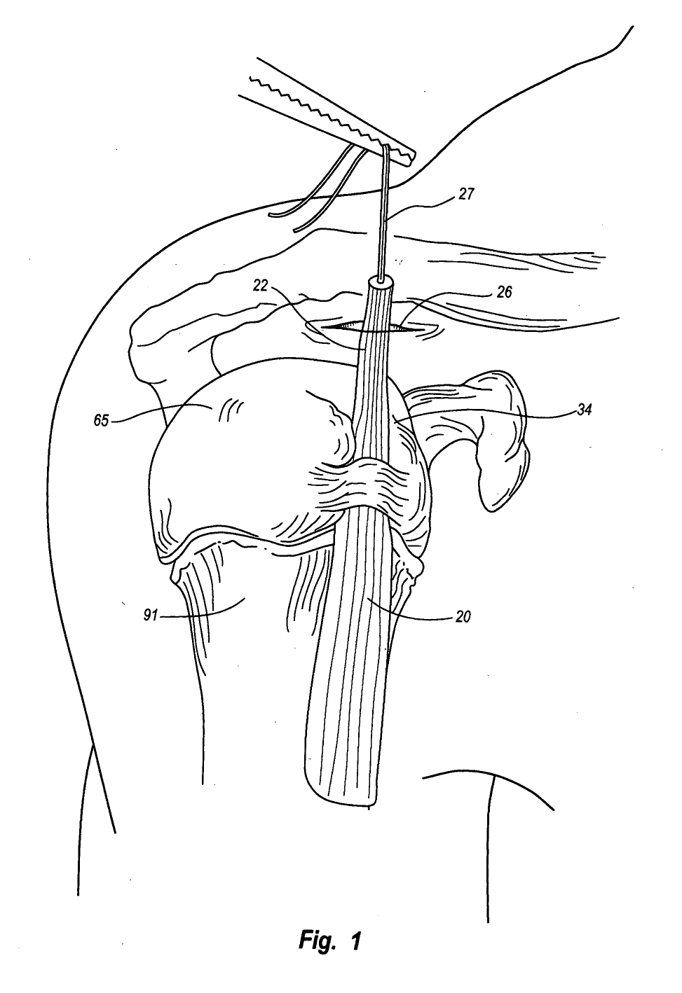

### DETAILED DESCRIPTION

Referring Now to the Drawings

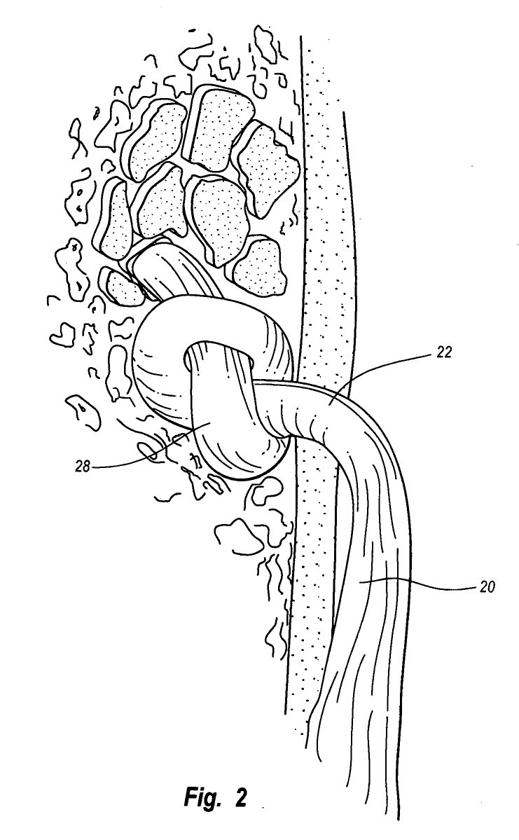

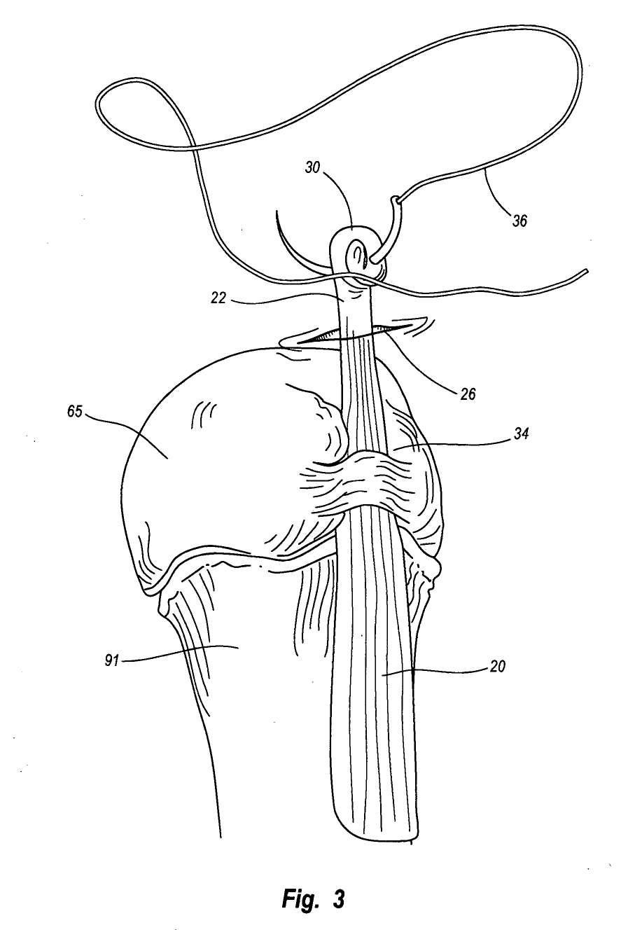

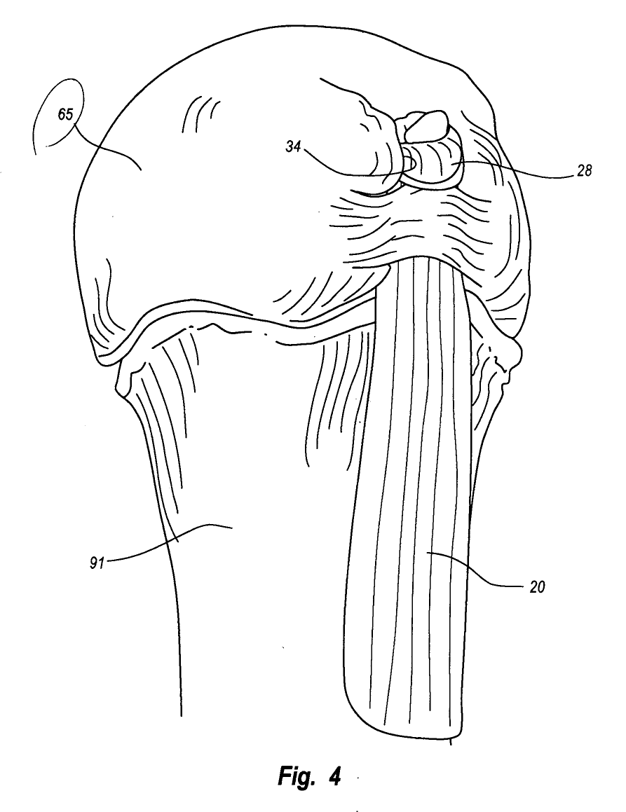

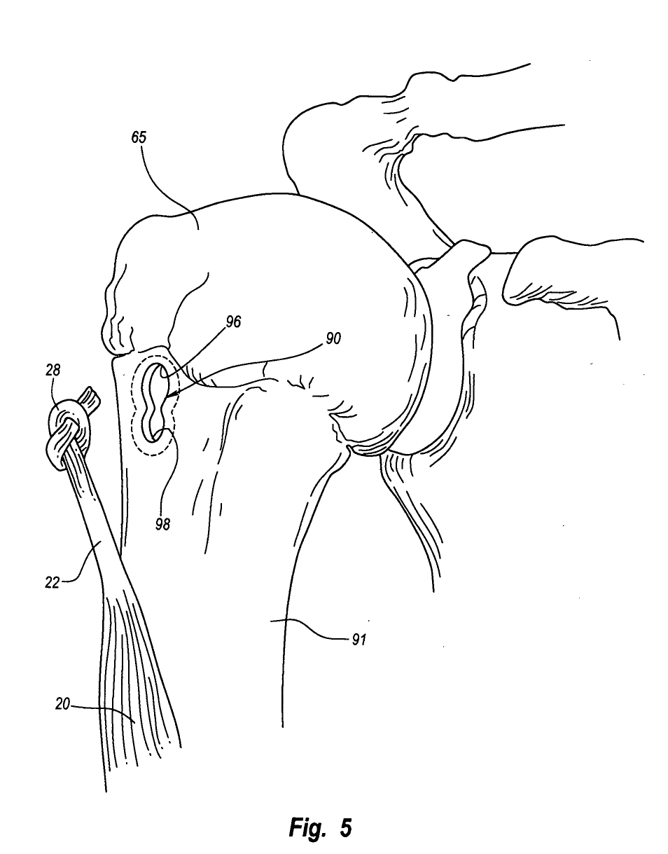

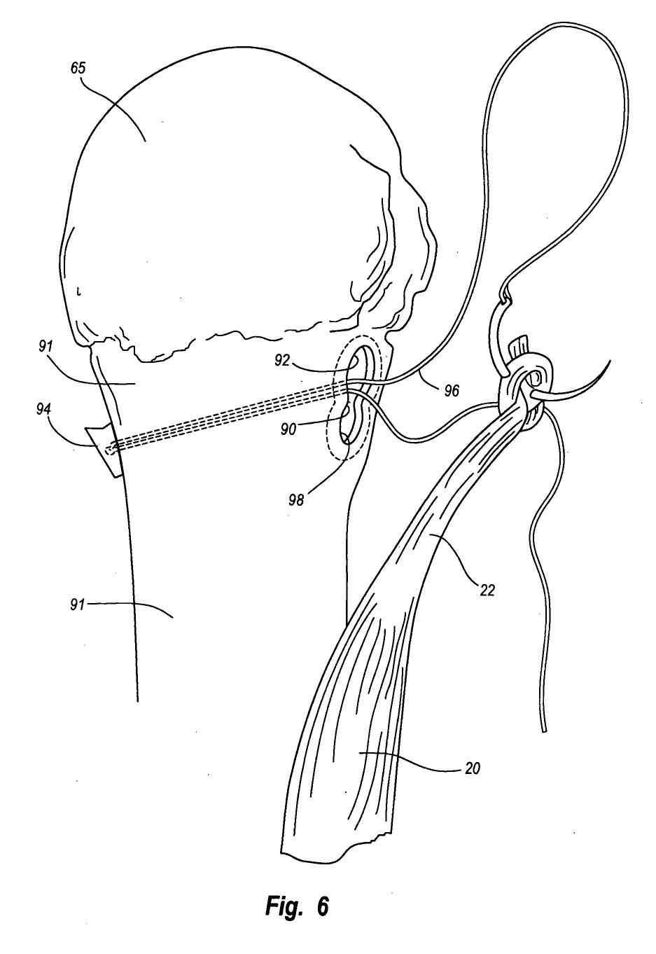

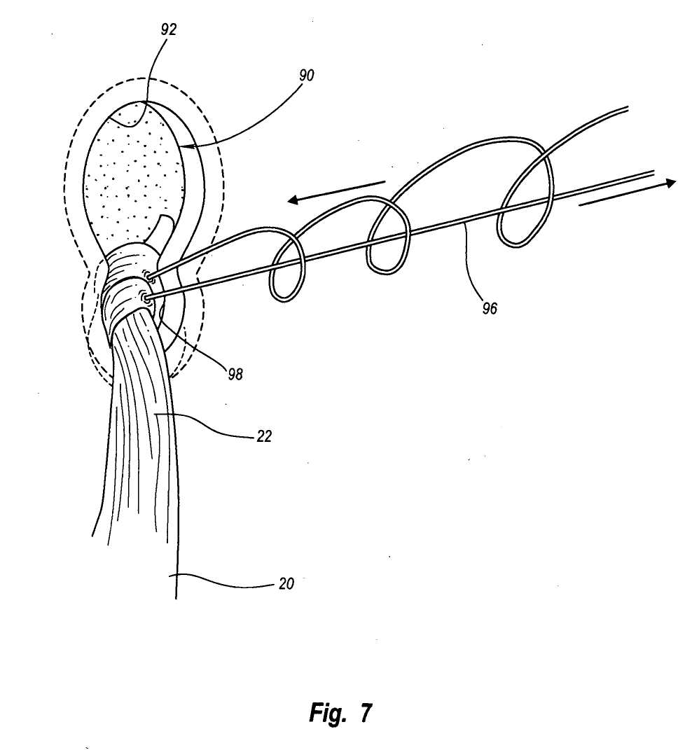

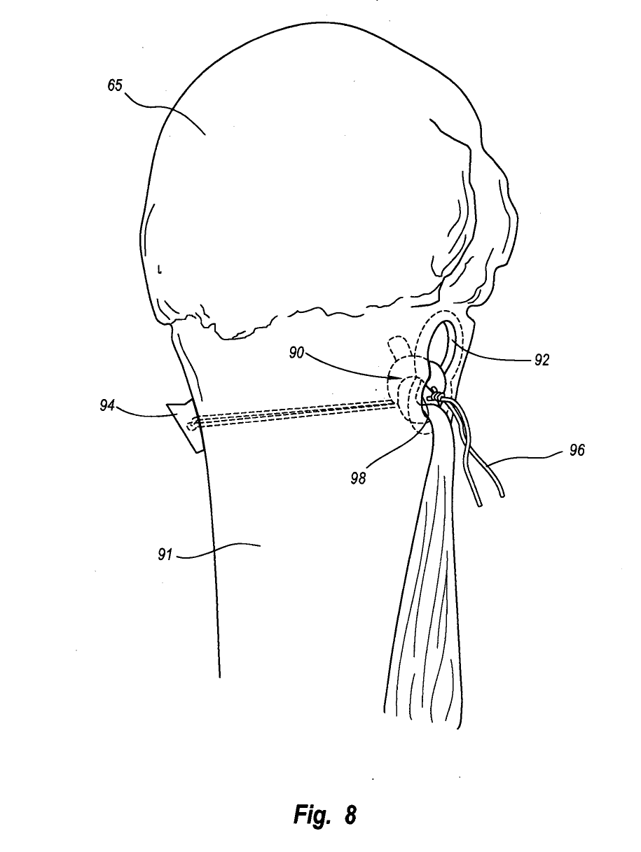

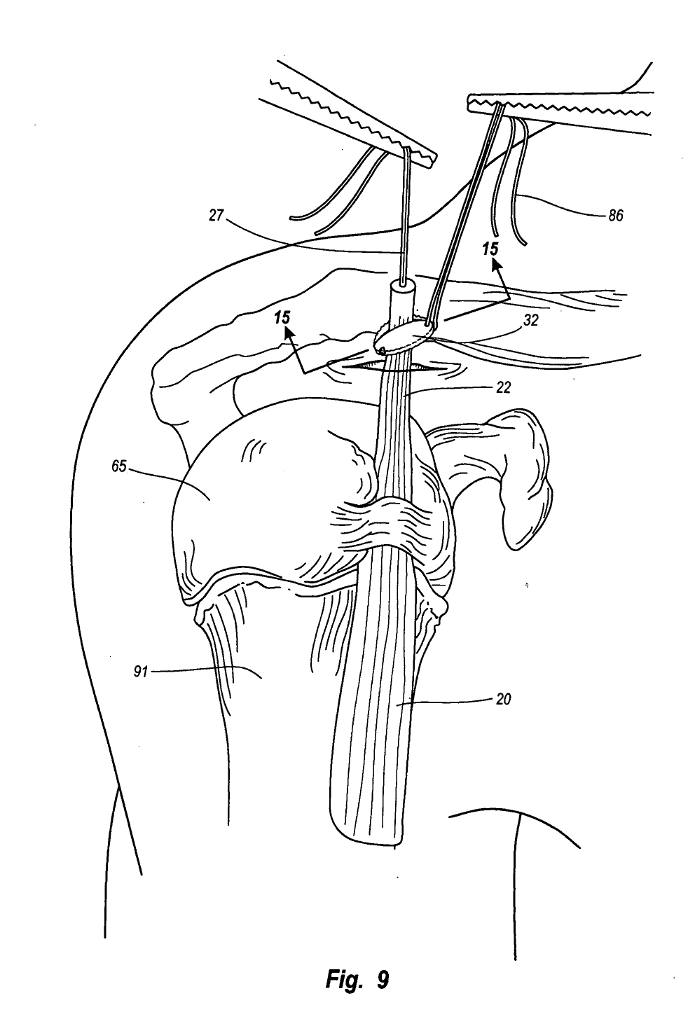

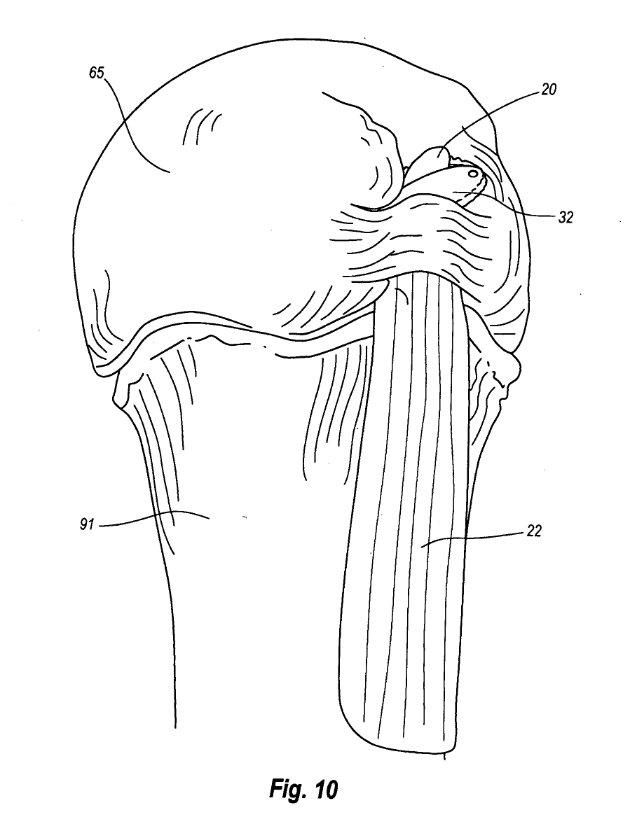

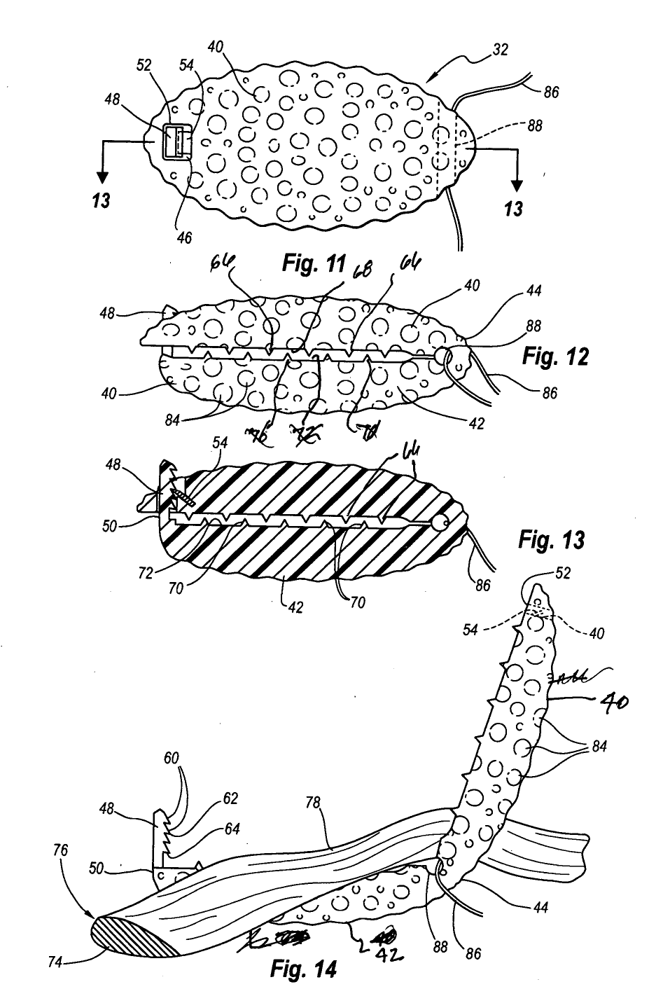

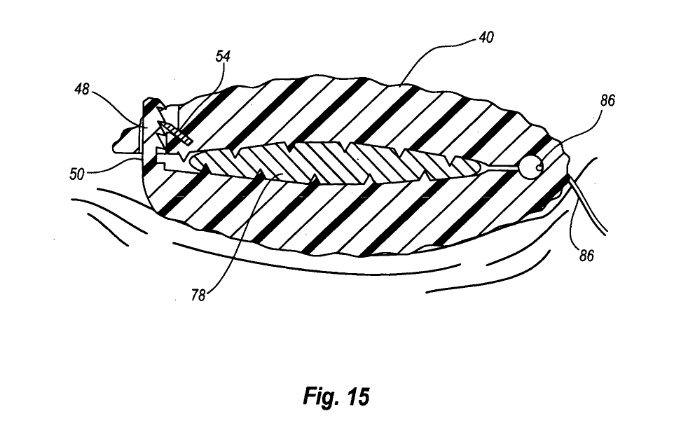

In a preferred embodiment, the process of wedge tenodesis disclosed involves the steps of (1) arthroscopically determining that a repair of a biceps tendon 20 is necessary using conventional arthroscopic procedures, and that a tenodesis is the best procedure for making such repair; (2) arthroscopically separating the head of the biceps tendon from the superior labium; (3) pulling the separated end 22 of the biceps tendon 20 through a portal 26 with a suture 27 and trimming the end 22 of the tendon 20; (4) selecting a wedge to be used from a tied knot wedge 28, FIG. 2, a sutured knot wedge 30, FIG. 3, or a clamp wedge 32, FIG. 9, to be affixed to the separated end 22 of the tendon 20; (5) affixing the selected wedge to the separated end 22 of the tendon 20; and (6) positioning the selected wedge in the bicipital groove 34 of the patient while allowing the biceps tendon 20 to retract within the bicipital groove 34 until held by engagement of the wedge with the top of the bicipital groove.

Selection of the wedge used from the tied knot wedge 28, the sutured knot wedge 30, or a button clamp wedge 32 will be based on recognized criteria and the experience of the surgeon. A tied knot wedge 28, if used, is affixed to the separated end 22 of the biceps tendon 20 by forming an overhand knot in the separated end of the biceps tendon. A tied knot will preferably be selected for use if the detached end of the biceps tendon is not severely frayed, torn or otherwise damaged. The sutured knot wedge 30 may be selected and used if the detached end 22 of the biceps tendon 20 is somewhat torn and frayed, but is in good enough condition to be rolled and secured in the rolled state by one or more sutures 36 placed through the rolled end.

The clamp wedge button 32 is selected when the surgeon performing the operation may lack sufficient experience with knot tying or suturing of a rolled end of the biceps tendon. The clamp wedge button is suitable for use as a replacement for either of the identified knot wedges, but is generally selected when the biceps end 22 is in good condition.

The clamp wedge button 32 is easier to install and has proven fully as effective in biceps tenodesis surgery as have either of the identified knot wedges. Consequently, the button wedge clamp may be preferred by even experienced surgeons, whenever a wedge tenodesis is performed and such a clamping wedge button is available for use.

Clamping wedge button 32 is preferably molded in one piece from a suitable plastic material, such as PLA plastic. The wedge is preferably formed with a top portion 40 and a bottom portion 42 interconnected by a hinge 44. The end of top portion 40 opposite the hinge 44 has a hole 46 formed therethrough. A rack 48 is formed to extend from the end of bottom portion 42 and is connected to the bottom portion by a hinge 50 that will allow the post to be pivoted towards the top portion 40 and to extend into hole 46 when the top portion 40 is pivoted at hinge 44 to overlie the bottom portion 42. The entrance 52 to hole 46 has a flange 54 extending inwardly thereof. Flange 54 extends angularly into hole 46 from the entrance 52.

Rack 48 has teeth 60 projecting from one side and spaced along the length of the rack 48. Each tooth 60 has an inclined guiding surface 62 extending from one side of the rack upwardly along the length of the rack. An abrupt holding surface 64 extends from the outer edge of each tooth normal to the rack 48.

Spaced apart parallel ribs 66 extend across the inner surface 68 of the top portion 40. Each rib has a triangular cross-sectional configuration with the apex of the rib extending into the space between top and bottom portions when the top portion 40 is folded over the bottom portion 42. Similar spaced apart ribs 70 are formed to extend across the inner surface 72 of bottom portion 42. The ribs 66 and 70 are spaced such that the ribs 66 on the top portion extend between and are spaced to be staggered with respect to the ribs 70 on the bottom portion when the top portion is pivoted to overlie the bottom portion.

In use, the clamping wedge button 32 is affixed to the separated end 22 of a biceps tendon, by positioning the bottom portion 42 of the clamp at one side 74 of the tendon 20 and pivoting the top portion 40 to extend over an opposite side 78 of the biceps tendon.

A locking rack 48 pivots from the bottom portion 42 and as the top and bottom portions are clamped together, with the biceps tendon between them, the rack extends into hole 46. Continued compression of the top and bottom portions 40 and 42 forces the free end of the locking rack 48 further into hole 46, with the engagement of the inclined surfaces 62 of the teeth 60 allowing insertion of the rack past the angled flange 54 extending into the hole 46. Engagement of abrupt surfaces 64 of the teeth with the flange 54 prevents withdrawal of the rack 48 from hole 46. The biceps tendon 20 is thus clamped securely between the top and bottom portions 40 and 42. The apex of the ribs 66 and 70 of the top and bottom portions 66 and 76 are pressed into the biceps tendon and the clamp is secured to the tendon.

In most instances it will be advantageous to have the clamping wedge button 32 extending diagonally across the clamped biceps tendon since this will allow the button to clamp to a significant portion of undamaged tendon and will reduce the possibility that the button clamp will cut or damage an already weakened section of the biceps tendon being repaired.

The button clamp wedge 32 with the top and bottom portions 40 and 42 clamped together has a general appearance of a football having all outer surfaces “roughened” as shown at 84 to provide for better holding action when the button is wedged into the bicipital groove 34.

When the button clamp wedge is used, the long head of the biceps tendon being separated is excised from the superior labrum, and is pulled through a cannula and a portal in the manner previously described in connection with the previously described knot wedge tenodesis.

A suture 86 is passed through the hole 88 provided at the hinge 44 between the top and bottom portions 44 and 42. The button clamp wedge 32 is clamped on the end 22 of the biceps tendon and the tendon, with button clamp wedge thereon is released to be guided, using the suture 86, into wedging position in the bicipital groove 34. If deemed necessary, the bicipital groove may be roughened prior to placement of the clamp wedge in the groove.

The wedge tenodesis provides for biceps tendon repair using known arthroscopic technology and avoidance of the incisions necessary for biceps tenotomy. Consequently, wedge tenodesis can be performed faster and without the same degree of surgical experience necessary to the repair of biceps tendons using other established repair procedures.

It is also clear that if a knot or button shall fail to wedge tightly in the bicipital groove, the wedge used, whether knot or button, can thereafter be alternatively fitted into and be secured in a keyhole slot 90 formed in the humeras 91. Should this become necessary, the keyhole slot 90 is formed in known fashion and with a bore hole 94 extending through the humeras in known fashion from the keyhole slot 90. The wedge, whether knot or button, has the suture line 86 passed therethrough. The knot or button is pulled into the large opening 92 of the keyhole, in known fashion, using an anchor 94 and suture 96 and is allowed to drop into the smaller opening 98 of the keyhole. The wedge is then held in the keyhole to secure the large head of the tendon.

Although preferred forms of our invention have been herein disclosed, it is to be understood that the present disclosure is by way of example and that variations are possible without departing from the subject matter coming within the scope of the following claims, which subject matter we regard as our invention.

1. A method for arthroscopic biceps tenodesis comprising
(a) arthroscopically determining that biceps tenodesis is desirable;
(b) arthroscopically separating the long head of the biceps tendon from attachment to a bone;
(c) creating and placing a wedge structure secured at the proximal end of the long head of the biceps tendon;
(d) allowing the tendon to slide through the glenohumerel joint; and
(e) lodging the wedge structure in the bicipital groove before the tendon slides fully through the glenohumerel joint.

2. A method as in claim 1, wherein
the long head of the biceps tendon is pulled through a portal to facilitate creating and placement of a wedge structure at the long head of the biceps tendon.

3. A method as in claim 2, wherein
a knot is tied in the long head of the biceps tendon as a wedge structure.

4. A method as in claim 2, wherein
the wedge structure is formed by rolling the long head of the biceps tendon and suturing said rolled head as a knot.

5. A method as in claim 2, wherein
the wedge structure formed is a button clamp secured to the long head of the biceps tendon.

6. A method as in claim 5, wherein
the button clamp has top and bottom portions and the long head of the biceps tendon is clamped between said top and bottom portions.

7. A method as in claim 6, wherein
the button clamp is secured diagonally across the length of the biceps tendon.

8. A button clamp for use in performing wedge tenodesis of a biceps tendon comprising
a generally football shaped body comprising a first top body portion and a second bottom portion;
a hinge interconnecting one end of said top body portion to an end of said bottom portion;
means for locking the other locking end of the top portion to the other locking end of the bottom portion;
gripping means extending from the top portion, each said gripping means having an apex extending towards said bottom portion when said portions are locked together; and
gripping means extending from the bottom portion, each said gripping means having an apex extending towards said top portion, when said portions are locked together said gripping means on said top portion and said gripping means on said bottom portion alternating along the length of said body.

9. A button clamp as in claim 8, wherein the means locking the other end of the top portion to the other end of the bottom portion, comprises
a rack having one end pivotally connected to the locking end of the button portion;
a receiving opening formed at the locking end of the top portion; and
means to secure an opposite end of the rack in the receiving opening.

10. A button clamp as in claim 9, wherein the means to secure an opposite end of the rack within the receiving opening comprises
a flange extending angularly into the receiving opening;
a plurality of spaced apart sloped teeth extending from the rack, each said tooth extending from the rack including an abrupt surface to engage the flange extending into the receiving opening to prevent withdrawal of the rack from the receiving opening.

11. A button clamp as in claim 10, wherein
the teeth on the rack and the flange extending into the receiving opening have sufficient flexibility to allow insertion of the opposite end of the rack and the teeth thereon into the receiving opening, and are sufficiently rigid to prevent withdrawal of the rack from the receiving opening.

12. A button clamp as in claim 8, whereby the gripping means is adapted to clamp on a tendon.

13. A button clamp as in claim 8, whereby the gripping means is adapted to clamp on a biceps tendon in a wedge tenodesis.

14. A button claim as in claim 8, whereby the gripping means is adapted to clamp on a tendon in an arthroscopic wedge tenodesis of a biceps tendon.
